(19) United States
(12) Patent Application Publication
(10) Pub. No.: US 2020/0346947 A1
(43) Pub. Date: Nov. 5, 2020

(54) Advanced Oxidation UV Sterilizer

(71) Applicant: Karin Smith, The Woodlands, TX (US)

(72) Inventor: Karin Smith, The Woodlands, TX (US)

(21) Appl. No.: 16/402,150

(22) Filed: May 2, 2019

(51) Int. Cl. C02F 1/32; C02F 1/72; C02F 1/78; A61L 9/20; A61L 9/015

(52) U.S. Cl. CPC C02F 1/32; C02F 1/725; C02F 1/78; A61L 9/20; A61L 2209/212; C02F 2305/10; C02F 2305/023; C02F 2201/3223; A61L 9/015

(57) ABSTRACT

A sterilizer apparatus for treating liquids or gasses, utilizing the low wavelength, ozone producing, vacuum UV wavelengths, utilizing a novel reactor chamber built around an ultraviolet lamp operating in the presence of either a vacuum or an oxygen-free medium.

### TECHNICAL FIELD

The present disclosure relates generally to Ultra Violet (UV) disinfection systems. More specifically, but not by way of limitation, this disclosure relates to treating mediums (gasses or liquids) using a novel internal reactor configuration designed to deliver all UV wavelengths, particularly the shorter wavelengths, to the medium in order to effect higher disinfection doses to the medium. Furthermore, the present disclosure relates to quartz sleeve surface generation of ozone and hydroxyl radicals in the medium contact region.

### CROSS REFERENCE TO RELATED APPLICATIONS

### U.S. Patent Documents

U.S. Pat. No. 5,393,419—Ultraviolet Lamp Assembly for Water Purification (February 1995)
U.S. Pat. No. 5,675,153—UV Apparatus for Fluid Treatment (October 1997)
U.S. Pat. No. 5,785,845—Water Purifying System (July 1998)
U.S. Pat. No. 6,344,176 B1—Device for Treating Liquids, Especially Coolants and Lubricants (February 2002)
U.S. Pat. No. 6,916,452 B1—Sterilization of Liquids Using Ultraviolet Light (July 2005)

### BACKGROUND OF THE INVENTION

In an Ultra Violet (UV) reactor, i.e. a simple or plain chamber with UV source(s), the UV source(s) typically have their quartz emitting regions doped; a coating method implemented by lamp manufacturers to prevent the shorter UV wavelengths (typically 187 nm) from being emitted together with the typically desired germicidal and longer UV wavelengths (typically 254 nm in low pressure lamps). The reason the shorter UV wavelengths are purposely blocked is to prevent the formation of ozone in the air pocket that surrounds the lamp inside the quartz sleeve. It is common knowledge that Ozone is a UV inhibitor so unwanted ozone in the quartz sleeve would inhibit the delivery of germicidal UV wavelengths from reaching the medium in the reactor beyond the quartz sleeve therefore rendering it ineffective for the purposes of UV disinfection. The short wavelengths of UV are therefore referred to as Vacuum UV (VUV) since they only exist in a vacuum. UV lamp manufacturers do produce un-doped versions of their germicidal lamps for the express purpose of generating ozone; in this application air is drawn across the un-doped lamp whereby the short UV wavelengths turn the available oxygen (O₂) in the passing airstream into ozone (O₃). The ozone is then typically injected into a medium intended for oxidation purposes.

Typical applications for fluid or gas disinfection by UV radiation are found in many industries as an effective method of inactivating bacteria, virus cysts and oocysts. This UV disinfection solution inactivates the pathogens with a non-chemical process.

Advanced Oxidation Process (AOP) is a method of combining either a chemical, e.g. hydrogen peroxide, or gas Ozone, with UV in a UV photo-catalytic process to form hydroxyl radicals. These OH radicals are then used to break up unwanted complex long-chain dissolved contaminants in a water source, example 1-4 Dioxane (carcinogen), so that they can be removed downstream of the AOP process. AOP treatment examples include ground water tables, wells and surface water sources contaminated by dissolved fertilizers, unused medications and industry related pollutants.

Since UV is a source of short wavelength electromagnetic energy; it is readily absorbed by the DNA of organisms (FIG. 2). The delivery of adequate UV energy (dose) to the DNA breaks up the double bond in pyrimidine bases (such as thymine and cytosine in DNA that hold the DNA structure together thus distorting the structure and halting the natural replication process of the organism. This DNA distortion process is called inactivation and is UV dose dependant, dose being defined as the product of UV intensity and exposure time and is expressed in the units of either J/m² or mJ/cm². In essence, the higher the dose, the more the DNA is distorted and damaged. A typical low pressure doped quartz lamp produces a monochromatic UV-C energy level output at 253.7 nm (254 nm). From FIG. 1 it can be seen that at this wavelength, referred to as the germicidal wavelength the energy absorption level of the organism's DNA is at approximately 100%, the peak, excluding VUV wavelengths, would be at 260 nm.

From the DNA absorption and sensitivity curves it can be seen that VUV wavelengths below 220 nm would yield significantly higher effective disinfection dose delivery to the organism's DNA, however under normal circumstances the generated UV inhibiting ozone would prevent this from occurring. The invention details the mechanism to achieve VUV energy delivery to the organisms' DNA without the production of ozone using un-doped UV lamps.

### BRIEF DESCRIPTION OF THE DRAWINGS

FIG. 1 represents a typical UV Reactor used for biological sterilization of a liquid using UV radiation.

FIG. 2 illustrates the typical microorganism's DNA sensitivity curve (200) and the germicidal spectral output of a doped quartz low pressure UV lamp (201). All energy absorbed within the sensitivity curve area (200) has a germicidal effect on the organism. For example it would take five times the amount of energy at approximately 290 nm to have the equivalent germicidal effect on an organism as one unit at 254 nm. All electromagnetic energy within the sensitivity curve (200) is accumulative in the DNA distortion process. In a polychromatic UV lamp (medium pressure UV lamp) where multiple wavelengths are emitted within the DNA sensitivity curve region, all these multiple energy outputs are accumulated at the inactivation levels depending on their wavelengths' into a total effective germicidal dose delivery. Although the peak energy absorption (160%) of the DNA occurs in the very short wavelengths around 200 nm, below 220 nm the wavelengths are trapped by the lamp manufacturers deliberate quartz emitter doping process to prevent the ozone formation (Germicidal UV inhibitor).

FIG. 3 illustrates the effective DNA absorption region (300) with a UV lamp that has doped quartz material to block all emissions below 220 nm. Any energy band emitted within the area below this curve (301) will have DNA germicidal distortion effects on the organism.

FIG. 4 illustrates the effective DNA absorption region (400) with a UV lamp that has un-doped quartz material that does not block any emissions below 220 nm. Any energy band emitted within the area below this curve (401) will have DNA germicidal distortion effects on the organism.

FIG. 5 represents the complete UV Reactor incorporating the novel structure which is the object of this invention. An externally sealed electrical connection (500) is provided as a means to make the electrical connection to the UV lamp (506). A sealed gas nipple (501) is provided as a means to extract the air within the internal parts of the reactor. A system of O-rings (502) is provided in order to maintain an external seal for the reactor and maintain the internal environment from external air ingress. A medium inlet pipe (503) and outlet pipe (505) join the main reactor body (504) and provide a medium flow path means through the reactor in order to effect disinfection on said medium. A quartz sleeve (507) provides an optical path and a mechanical barrier between the medium (gas or fluid) and the internal structure of the reactor. A system of O-rings (508) provides sealing between the quartz sleeve (507) and the reactor (504) to prevent medium ingress into the internal reactor system.

FIG. 6 represents the medium (601) flow path (602) through the reactor (600).

FIG. 7 represents the air extraction (702) pathway through the gas nipple, attached to the UV reactor (700) which causes a reactor internal structure vacuum (701). The oxygen depleted (vacuum) area (703) between the UV lamp and quartz sleeve (704) forms the basis of the novel invention.

FIG. 8 represents an alternative method for providing an oxygen depleted internal reactor environment. At least one gas nipple is used to allow an oxygen free medium to enter the reactors' (800) internal air purged environment, or a second gas nipple is used to allow the medium flow path (801) to enter the reactors' internal environment and then leave at a remote point (802). The methodology of the medium flow path will be to exhaust the ambient oxygen rich air inside the reactor and replace it with an oxygen free medium, nitrogen for example. The absence of oxygen in the internal region of the reactor between the lamp and quartz sleeve forms the basis of this invention.

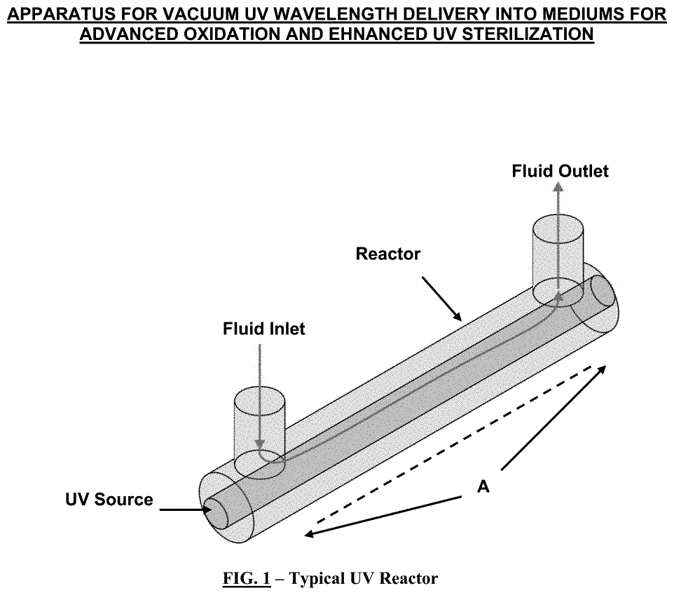

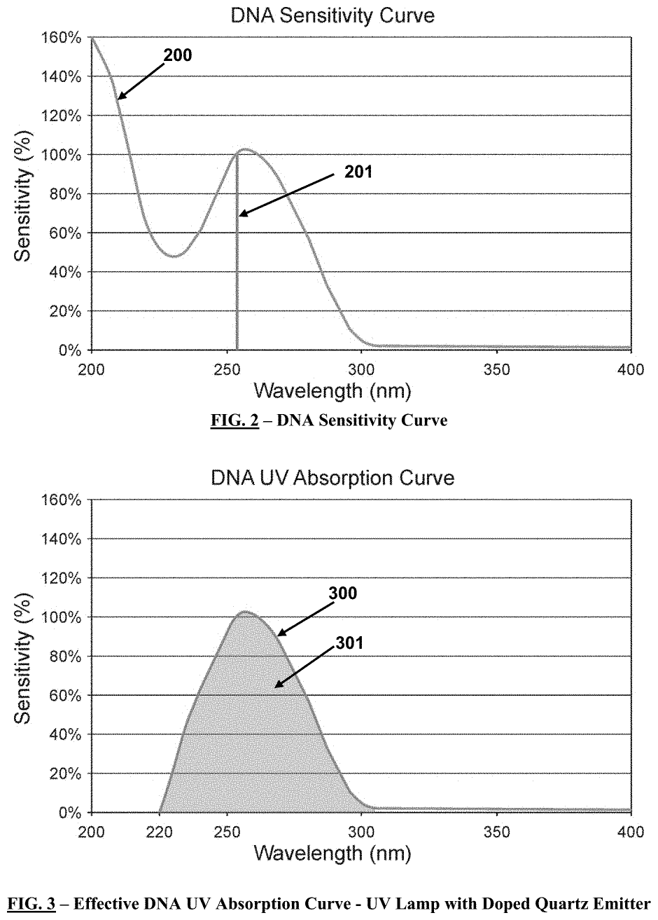

### DETAILED DESCRIPTION OF THE INVENTION

Certain aspects and features of the present disclosure relate to a UV sterilization apparatus including an oxygen depleted internal environment to enhance the disinfection process by making it highly efficient with the use of low wavelength producing UV lamps.

Additional aspects relate to the low UV wavelength production of hydroxyl radicals at the surface of the quartz sleeve where the medium, with dissolved oxygen, contacts it. The low wavelength UV electromagnetic energy dissociates the dissolved oxygen (O₂) in the medium, typically but not limited to water, and forms unstable Hydroxyl (OH) radicals. The OH radicals scavenge and oxidize unwanted and complex dissolved contaminants. This feature forms the method of the novel Advanced Oxidation Process (AOP) of this invention. The interaction of OH radicals and the germicidal UV energy form the photo-catalytic basis of UV-AOP.

In order to utilize all of the electromagnetic energy produced by a non-doped quartz emitter UV lamp, all the air around it must be depleted to form a vacuum.

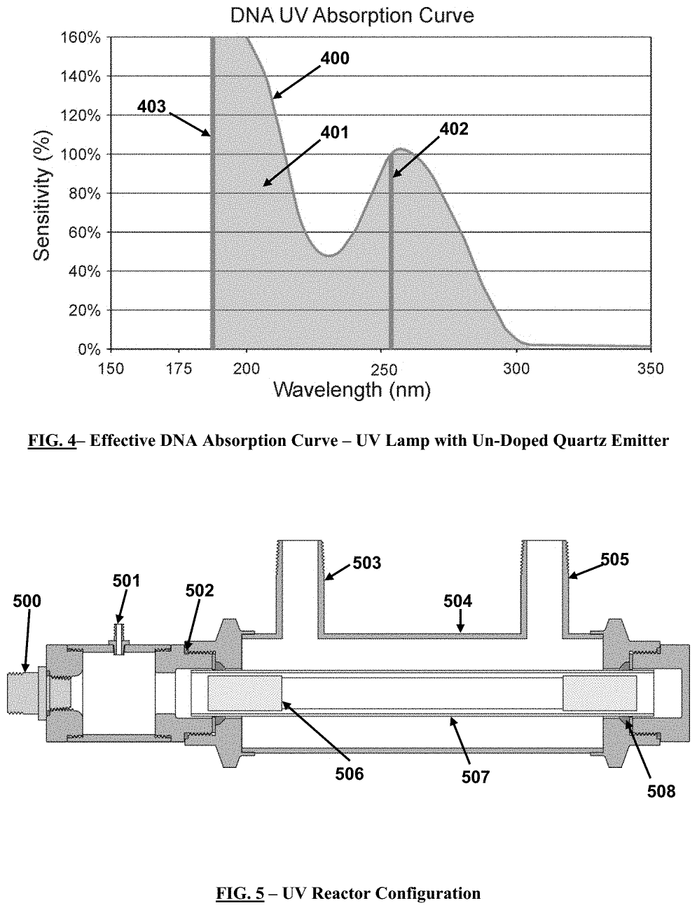

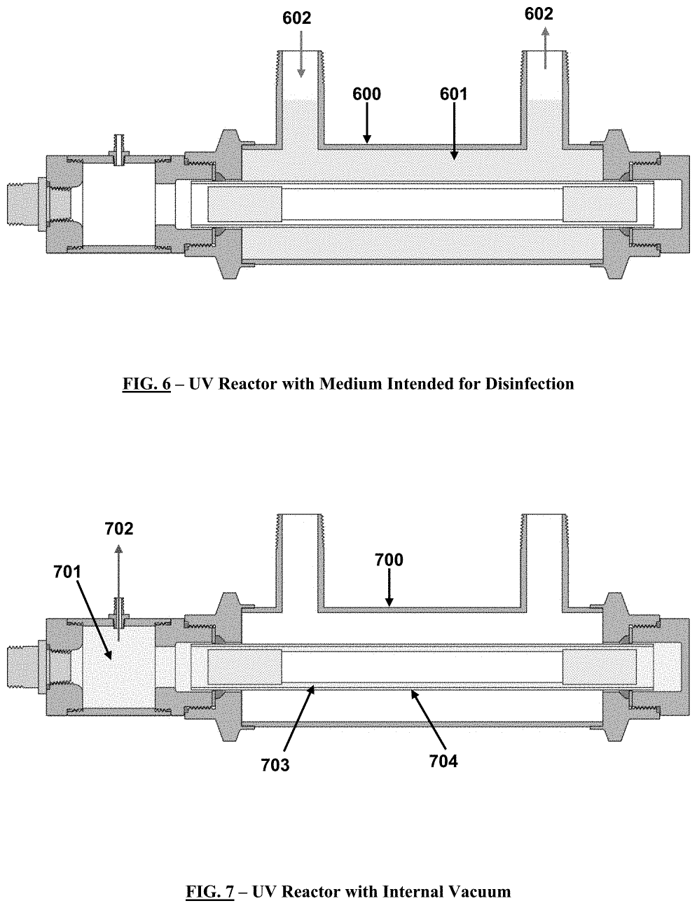

FIG. 4 represents the energy absorption curve of DNA; from this it is clear that the lower UV wavelengths have the highest potential germicidal effect (DNA distortion) on the pathogens', however under circumstances, outside the operation of this invention, in an environment where an un-doped quartz emitter UV lamp surrounded by oxygen is used, ozone (O₃) is produced by the sub-200 nm UV wavelengths. Ozone forms a natural UV barrier which envelops the area between the lamp and quartz sleeve and prevents (shields) the low and germicidal UV wavelengths from reaching the medium to be disinfected.

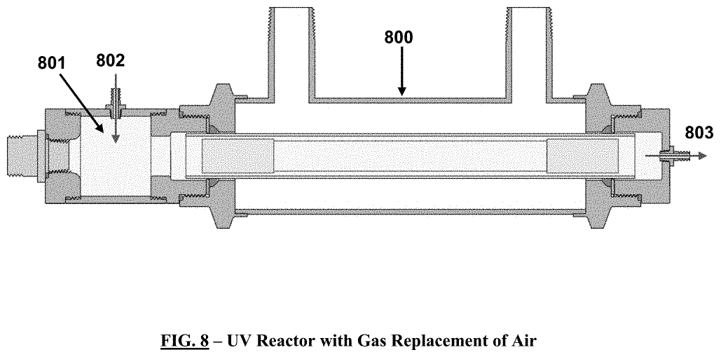

In this disclosure, either by internal reactor air removal (FIG. 7), or replacement of surrounding air, with an oxygen free medium (FIG. 8); all the UV wavelengths which are emitted from un-doped quartz emitter style UV lamps are able to travel freely to and beyond the quartz sleeve that surrounds it.

What is claimed is:

1. A method to enhance the disinfection process of an intended medium and make disinfection more efficient by using low wavelength vacuum UV (VUV) devices, which comprises:
a. an inner germicidal un-doped quartz emitter ultraviolet lamp as source of UV radiation;
b. an elongated quartz sleeve, extending around the ultraviolet lamp to protect the lamp to come into contact with the medium under treatment;
c. an outer tubular duct (UV reactor) containing an inlet and an outlet port at or close to its opposite ends to allow for easy flow of the medium under treatment constructing the UV reactor chamber; and,
d. an internal reactor environment free of oxygen.

2. The medium treatment apparatus described in claim 1 further utilizing the oxygen dissociation ozone production process properties of VUV wavelengths in the intended medium, typically, but not limited to, water, for the purposes of enhanced oxidation based disinfection and/or contaminant destruction set up by the UV photo-catalytic hydroxyl radical (OH) based advanced oxidation process (UV-AOP).
